(19) United States
(12) Patent Application Publication
(10) Pub. No.: US 2002/0123681 A1
(43) Pub. Date: Sep. 5, 2002

(54) Device and a system for moving and positioning an open magnetic resonance imaging probe

(76) Inventors: Yuval Zuk, Haifa (IL); Yoav Katz, Rehovot (IL); Avinoam Livni, Haifa (IL); Ruth Ben Kish, Givat-Elah (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 09/995,823

(22) Filed: Nov. 29, 2001

Related U.S. Application Data
(63) Continuation of application No. 09/443,867, filed on Nov. 19, 1999.
(60) Provisional application No. 60/111,395, filed on Dec. 8, 1998.

(51) Int. Cl. A61B 5/05

(52) U.S. Cl. 600/410

(57) ABSTRACT

A positioning device for positioning or an open MRI magnetic probe including an open MRI magnet in a plurality of positions and for controllably moving the probe between these positions. The positions include at least one imaging position of the open probe which allows the performing of magnetic resonance imaging procedures and surgical procedures on a body part, either sequentially or simultaneously, without moving the probe from the imaging position. The plurality of positions may include one or more positions in which the probe is retracted away from the body part to Increase the accessibility of the body part and to allow for use of magnetic field sensitive devices to be positioned near the body part. The positioning device includes a movable framework attachable to the probe and a position control unit operatively connected to the movable framework for controlling the moving of the framework. The framework may be motorized but may also include manually movable parts. The device is capable of moving the probe from one position to another with one or more degrees of freedom. The device may be adapted for repeatedly and accurately positioning the probe selected positions. Magnetic resonance imaging systems including the positioning device are also disclosed.

This application is a continuation application of U.S. patent application Ser. No. 09/443,867 filed Nov. 19, 1999, which claims the benefit of U.S. Provisional Application Serial No. 60/111,395 filed Dec. 8, 1998.

### FIELD OF THE INVENTION

The present invention relates generally to the field of apparatus for magnetic resonance imaging (MRI) and magnetic resonance therapy (MRT), and interventional magnetic resonance imaging (iMRI) and more specifically to devices and systems for moving and positioning open magnetic apparatus for performing MRI, MRT and iMRI.

### BACKGROUND OF THE INVENTION

MRI systems for performing whole body imaging usually employ large magnets that effectively surround the patient. Such magnets are usually superconductor magnets, which are large, do not provide access to the patient, and require a dedicated room. Generally, the design of such magnets involves a trade-off between the size of the field of view and the size of the magnet. The larger the magnet, the larger the field of view. However, when only local imaging of small sections of the body are required, it becomes possible to employ much more compact moveable systems with smaller magnets. For Interventional MRI (iMRI), only the area to be operated on is imaged, therefore a limited field of view is not a disadvantage.

Large superconducting magnets are expensive, and have high operating and maintenance costs. Such large superconducting magnets also require a dedicated room due to their large size and the strength of the magnetic field they produce. The use of large superconducting magnets in iMRI applications is also encumbered by sterilization problems.

Thus, while the use of iMRI in the operating theatre for providing the surgeon with realtime imaging feedback about the progress of surgical procedures may have many advantages, it is usually unavailable to surgeons in the operating room due to the limitations described above.

MRI systems utilizing permanent magnets are less expensive, do not require expensive cryogenic cooling systems, and do not require dedicated rooms due to their compact size, and lower magnetic field. Such systems can also be more easily sterilized.

U.S. Pat. No. 5,735,278 to Hoult et al. discloses an MRI system including a massive superconducting magnet which is moveable between a first position spaced from a bed carrying the patient which allows a surgical procedure to be carried out on a patient and a second position for applying a magnetic field to a part of the patient. To operate in the second position, the entire cylinder-like superconducting magnet has to be moved from the first position towards the operating table on which the patient is lying and positioned such that the part of the patient which is to be imaged and at least some of the operating table are disposed within the superconducting magnet's inner cylindrical bore. This has a disadvantage of blocking visual and physical access to the patient during the imaging time and during the time periods required for moving the magnet. The superconducting magnet probe is closed and must therefore be fully withdrawn after imaging to allow the surgeon access to the operated organ or body part. Another disadvantage of the system is that the large superconducting magnet is difficult to sterilize.

Other iMRI systems known in the art, such as the iMRI system disclosed in U.S. Pat. No, 5,365,927 to Roemer et al. have large fixed magnets having the disadvantages of preventing full access to the operating table and to the operated area during imaging and of making sterilization more difficult.

Prior art systems having large fixed magnets or large movable magnets have additional disadvantages. For example, full withdrawal of the probe from around the patient in movable magnet systems or full withdrawal of the patient bed outside of the magnet in systems with fixed magnets requires a relatively long time resulting in lengthening of the total operation time, and prevents the surgeon from obtaining realtime images while the surgical procedure is being performed. Additionally, the use of large fixed magnets has the disadvantage of exposing the surgical team to a strong magnetic field throughout the course of surgery.

Recently, small compact MRI and iMRI systems have been developed for intra-operative use. U.S. patent application Ser. No. 08/898,773 to Katznelson et al., filed Jul. 23, 1997, now U.S. Pat. No. 5,900,793, assigned to the assignee of the present application, titled “PERMANENT MAGNET ASSEMBLIES FOR USE IN MEDICAL APPLICATIONS”, the entire specification of which is incorporated herein by reference, discloses, inter alia, an MRI system having an open magnet including two spaced apart permanent magnet assemblies.

Co-pending U.S. Patent Application to Katznelson et al., filed Sep. 27, 1999, titled “YOKED PERMANENT MAGNET ASSEMBLIES FOR USE IN MEDICAL APPLICATIONS”, the entire specification of which is incorporated herein by reference, discloses, inter alia, an MRI system having an open magnet including two spaced apart permanent magnet assemblies attached to an open ferromagnetic yoke.

Co-pending U.S. patent application Ser. No. 09/161,336 to Zuk et al., filed Sep. 25, 1998, co-assigned to the assignee of the present application, titled “MAGNETIC APPARATUS FOR MRI”, the entire specification of which is incorporated herein by reference, discloses an iMRI system having an open magnet including two spaced apart permanent magnet assemblies having an open region therebetween and having gradient coils positioned outside the open region,

Co-pending U.S. patent application Ser. No. 09/295,814 to Panfil et al., filed Feb. 9, 1998, co-assigned to the assignee of the present application, titled “A METHOD FOR DESIGNING OPEN MAGNETS AND OPEN MAGNETIC APPARATUS FOR USE IN MRI/MRT PROBES”, the entire specification of which is incorporated herein by reference , discloses, inter alia, a open iMRI magnet having an open ferromagnetic yoke and including two spaced apart permanent magnet assemblies having ferromagnetic collimators arranged to have an open region therebetween.

Co-pending U.S. patent application Ser. No. 09/274,671 to Katznelson et al., filed Mar. 24, 1999, co-assigned to the assignee of the present application, titled “HYBRID MAGNETIC APPARATUS FOR USE IN MEDICAL APPLICATIONS”, the entire specification of which is incorporated herein by reference, discloses, inter alia, a compact open iMRI magnet having combined permanent magnet assemblies and electromagnet assemblies.

### SUMMARY OF THE INVENTION

There is therefore provided, in accordance with a preferred embodiment of the present invention, a system for magnetic resonance imaging. The system includes an operating table for supporting a patient, a magnetic resonance imaging system including an open magnetic probe for producing a volume of substantially homogenous magnetic field for imaging a body part of the patient, and a controllable positioning device attached to the open magnetic probe for controllably positioning the open magnetic probe in a position selected from a plurality of probe positions. The plurality of positions includes at least one imaging position in which the open magnetic probe is positioned relative to the body part of the patient such that surgical procedures and imaging procedures can be performed on the body part of the patient while the open magnetic probe is positioned in the at least one imaging position.

Furthermore, in accordance with another preferred embodiment of the present invention, the positioning device includes a controllably movable framework attached to the open magnetic probe and a position control unit operatively connected to the movable framework for controlling the moving of the open magnetic probe by the movable framework from one position of the plurality of probe positions to another position of the plurality of probe positions.

Furthermore, in accordance with another preferred embodiment of the present invention, the movable framework is a motorized framework including at least one motor operatively coupled to the framework for moving the magnetic probe relative to the operating table with at least one degree of freedom.

Furthermore, in accordance with another preferred embodiment of the present invention, the position control unit includes a microprocessor in communication with a data storage unit for retrievably storing data representative of the plurality of probe positions and for controlling the moving of the magnetic probe into a probe position selected from the plurality of probe positions.

Furthermore, in accordance with another preferred embodiment of the present invention, the open magnetic probe is selected from a U-shaped open magnetic probe, a C-shaped open magnetic probe and a Y-shaped open magnetic probe.

Furthermore, in accordance with another preferred embodiment of the present invention, the open magnetic probe is selected from an open magnetic probe including permanent magnets, an open yoked magnetic probe including permanent magnets, an open yoked magnetic probe including permanent magnets a ferromagnetic yoke and ferromagnetic collimators, an open magnetic probe including electromagnets, and a hybrid open magnetic probe including permanent magnets and electromagnets.

Furthermore, in accordance with another preferred embodiment of the present invention, the electromagnets are selected from resistive electromagnets and superconducting electromagnets.

Furthermore, in accordance with another preferred embodiment of the present invention, the positioning device is detachably attached to the operating table.

Furthermore, in accordance with another preferred embodiment of the present invention, the operating table is positioned in an operating room, and the positioning device is attached to a part of the operating mom, the part is selected from the ceiling of the operating room, at least one wall of the operating room, the floor of the operating room and any combination thereof.

Furthermore, in accordance with another preferred embodiment of the present invention, each position of the plurality of probe positions is defined relative to a position selected from the position of the operating table and the position of the body part.

Furthermore, in accordance with another preferred embodiment of the present invention, the positioning device is a transportable positioning device.

Furthermore, in accordance with another preferred embodiment of the present invention, the plurality of probe positions includes at least one probe position in which at least part of the magnetic probe is disposed under the operating table.

Furthermore, in accordance with another preferred embodiment of the present invention, the plurality of probe positions includes at least two probe positions suitable for performing magnetic resonance imaging of the body part to obtain at least two different partially overlapping images of the body part, the two images are suitable for being processed to obtain therefrom a composite image having an image volume larger than the image volume of each of the two partially overlapping images.

There is also provided, in accordance with another preferred embodiment of the present invention, a position controlling device for use in a magnetic resonance imaging system to obtain magnetic resonance images of a body part of a patient using an open magnetic probe. The device includes a controllably movable framework attachable to the open magnetic probe for controllably positioning the open magnetic probe in a position selected from a plurality of probe positions The plurality of positions includes at least one imaging position in which the open magnetic probe is positioned relative to the body part of the patient such that imaging procedures and surgical procedures can be performed on the body part of the patient while the open magnetic probe is positioned in that imaging position. The position controlling device further includes a position control unit operatively connected to the movable framework for controlling the moving of the open magnetic probe by the movable framework from one position of the plurality of probe positions to another position of the plurality of probe positions.

Furthermore, in accordance with another preferred embodiment of the present invention, the movable framework is detachably attachable to an operating table supporting the patient.

Furthermore, in accordance with another preferred embodiment of the present invention, the plurality of probe positions includes at least one probe position in which the magnetic probe is at least partially disposed under the operating table.

Furthermore, in accordance with another preferred embodiment of the present invention, the operating table is positioned in an operating room, and the positioning device is attached to a part of the operating room. The part is selected from the ceiling of the operating room, at least one wall of the operating room, the floor of the operating room and any combination thereof.

Furthermore, in accordance with another preferred embodiment of the present invention, each position of the plurality of probe positions is defined relative to a point positioned on the operating table and a point on or within the body part. The body part is attached to or disposed upon the operating table.

Furthermore, in accordance with another preferred embodiment of the present invention, each position of the plurality of probe positions is defined relative to a point on or within the body part.

Furthermore, in accordance with another preferred embodiment of the present invention, the plurality of probe positions includes at least two probe positions suitable for performing magnetic resonance imaging of the body part to obtain at least two different partially overlapping images of the body part. The two partially overlapping images are suitable for being processed to obtain therefrom a composite image having an image volume larger than the image volume of each of the two partially overlapping images.

Furthermore, in accordance with another preferred embodiment of the present invention, the movable framework is a motorized framework including at least one motor operatively coupled to the framework for moving the magnetic probe with at least 1 degree of freedom.

Furthermore, in accordance with another preferred embodiment of the present invention, the position control unit includes a microprocessor in communication with a data storage unit for retrievably storing data representative of the plurality of probe positions and for controlling the moving of the magnetic probe into a probe position selected from the plurality of probe positions.

Furthermore, in accordance with another preferred embodiment of the present invention, each position of the plurality of probe positions is defined relative to the position of the body part.

There is also provided, in accordance with yet another preferred embodiment of the present invention, a controllably movable magnetic probe device for use in a magnetic resonance imaging system for obtaining magnetic resonance images of a body part. The magnetic probe device includes an open magnet for producing a volume of substantially homogenous magnetic field for imaging a body part. The magnet is adapted for being disposed in at least one imaging position relative to the body part such that surgical procedures and imaging procedures can be sequentially and/or simultaneously performed on the body part while the open magnet is positioned in the imaging position. The magnetic probe device further includes a controllable positioning device attached to the open magnet for controllably positioning the open magnet in a position selected from a plurality of magnet positions. The plurality of positions includes the imaging position.

Furthermore, in accordance with another preferred embodiment of the present invention, each position of the plurality of magnet positions is defined relative to a point on or within the body part.

Furthermore, in accordance with another preferred embodiment of the present invention, the plurality of magnet positions includes at least two magnet positions suitable for performing magnetic resonance imaging of the body part to obtain at least two different partially overlapping images of the body part. The two partially overlapping images are suitable for being processed to obtain therefrom a composite image having an image volume larger than the image volume of each of the two partially overlapping images.

Furthermore, in accordance with another preferred embodiment of the present invention, the movable framework is a motorized framework including at least one motor operatively coupled to the framework, for moving the magnet with at least 1 degree of freedom.

Furthermore, in accordance with another preferred embodiment of the present invention, each position of the plurality of magnet positions is defined relative to the position of the body part.

Furthermore, in accordance with another preferred embodiment of the present inventions the position control unit is adapted to restrict the moving of the magnetic probe within an allowed spatial envelope.

Furthermore, in accordance with another preferred embodiment of the present invention, the allowed spatial envelope is defined with respect to an operating table attached to the controllable positioning device.

Furthermore, in accordance with another preferred embodiment of the present invention, the positioning device is a transportable positioning device.

Furthermore, in accordance with another preferred embodiment of the present invention, the positioning device includes a controllably movable framework attached to the open magnet and a position control unit operatively connected to the movable framework for controlling the moving of the open magnet by the movable framework from one position of the plurality of magnet positions to another position of the plurality of magnet positions.

Furthermore, in accordance with another preferred embodiment of the present invention, the movable framework is attached to a part of an operating room, the part is selected from the ceiling of the operating room, at least one wall of the operating room, the floor of the operating room and any combination thereof.

Furthermore, in accordance with another preferred embodiment of the present invention, the position control unit includes a microprocessor in communication with a data storage unit for retrievably storing data representative of the plurality of magnet positions and for controlling the moving of the magnet into a magnet position selected from the plurality of magnet positions

Furthermore, in accordance with another preferred embodiment of the present invention, the movable framework is detachably attachable to an operating table.

Furthermore, in accordance with another preferred embodiment of the present invention, each position of the plurality of magnet positions is defined relative to a point positioned on the operating table and a point on or within the body part, the body part being attached to or disposed upon the operating table.

Furthermore, in accordance with another preferred embodiment of the present invention, the plurality of magnet positions includes at least one magnet position in which the magnet is at least partially disposed under the operating table.

Finally, in accordance with another preferred embodiment of the present invention, the operating table is positioned in an operating room, and the positioning device is attached to a part of the operating room, the part is selected from the ceiling of the operating room, at least one wall of the operating room, the floor of the operating room and any combination thereof.

### BRIEF DESCRIPTION OF THE DRAWINGS

The invention is herein described, by way of example only, with reference to the accompanying drawings, in which like components are designated by like reference numerals, wherein:

FIG. 1 is a schematic isometric view of a positioning device for intra-operative positioning of an open MRI probe in an iMRI system, in accordance with a preferred embodiment of the present invention;

FIG. 2 is a schematic exploded view illustrating a system for interventional magnetic resonance imaging (iMRI) including an open magnetic probe, a positioning device and an operating table, in accordance with a preferred embodiment of the present invention;

FIG. 3 is a schematic isometric view illustrating the iMRI system of FIG. 2, with the positioning device and MRI probe of FIG. 1 in an imaging position;

FIG. 4 is a schematic isometric view illustrating the iMRI system of FIG. 2, with the positioning device and the MRI probe of FIG. 1 in a retracted position under the operating table;

FIG. 5 is a schematic functional block diagram illustrating a position control unit for an iMRI system, in accordance with a preferred embodiment of the present invention;

FIGS. 6 and 7 are schematic isometric views illustrating a marker device for positioning a magnetic probe in a desired imaging position, in accordance with a preferred embodiment of the present invention;

FIG. 8 is a schematic isometric view illustrating an iMRI system having a ceiling-mounted positioning device for intra-operative positioning of an open MRI probe, in accordance with another preferred embodiment of the present invention;

FIG. 9 is a schematic isometric view illustrating the iMRI system of FIG. 8, with the MRI probe in an imaging position; and

FIG. 10 is a schematic isometric view of a positioning device for intra-operative positioning of an open MRI probe in an iMRI system, adapted for moving the MRI probe with one degree of freedom, in accordance with another preferred embodiment of the present invention.

### DETAILED DESCRIPTION OF THE INVENTION

### Notation Used Throughout

The following notation is used throughout this document.

| Term | Definition |
|---|---|
| MRI | Magnetic Resonance Imaging |
| MRT | Magnetic Resonance Therapy |
| iMRI | Interventional Magnetic Resonance Imaging |
| FOV | Field of View |

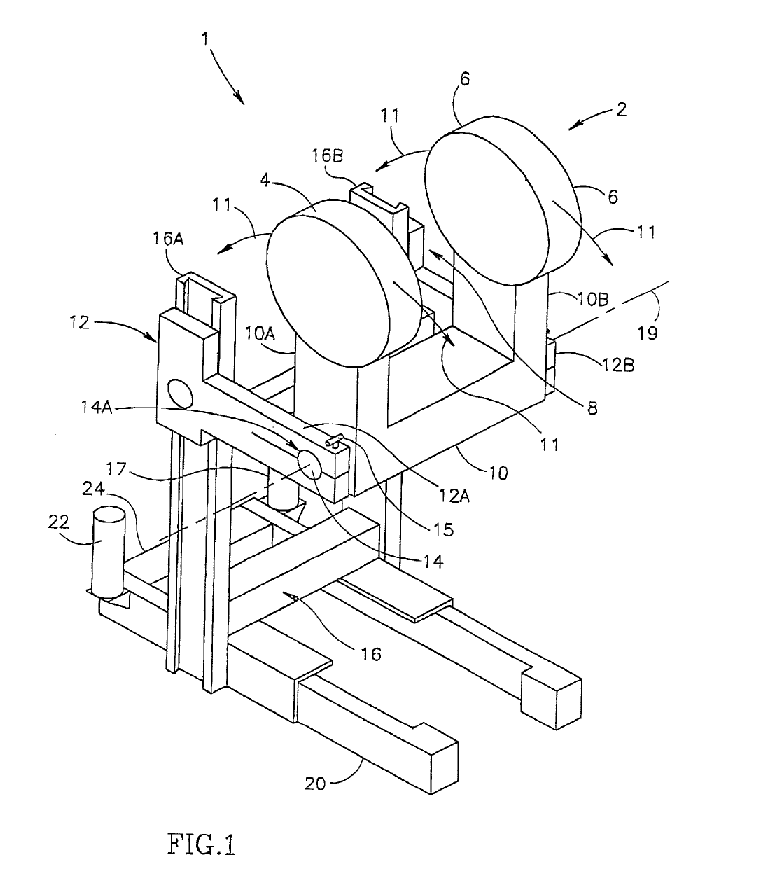

Reference is now made to FIG. 1 which is an isometric view of a positioning device for intra-operative positioning of an open MRI probe in an iMRI system, in accordance with a preferred embodiment of the present invention. The device 1 is attachable to an operating table (not shown) and to an open MRI probe 2. The MRI probe 2 includes two opposing magnet assemblies 4 and 6 having an open region 8 therebetween. The magnet assemblies 4 and 6 are attached to two arms 10A and 10B, respectively, of a U-shaped member 10. The structure and the magnetic design of the magnet assemblies 4 and 6 are not the subject matter of the present invention. The magnet assemblies 4 and 6 may be the permanent magnet assemblies disclosed in U.S. Pat. No. 5,900,793 to Katznelson et al., U.S. patent application Ser. No. 09/161,336 to Zuk et al., U.S. patent application to Katznelson et al., filed Sep. 27, 1999, entitled “YOKED PERMANENT MAGNET ASSEMBLIES FOR USE IN MEDICAL APPLICATIONS”, U.S. patent application Ser. No. 09/295,814 to Panfil et al., and U.S. patent application Ser. No. 09/274,671 to Katznelson et al. referenced hereinabove. However, the magnet assemblies 4 and 6 may also be any other suitable magnet assemblies having an open region therebetween, including but not limited to permanent or super-conducting or resistive electromagnet assemblies or hybrid magnetic apparatus including a combination of permanent magnets and electromagnets.

The U-shaped member 10 is rotatably attached to a vertically movable framework 12 by two cylindrical shafts 14 rigidly attached to the U-shaped member 10. The framework 12 includes two arms 12A and 12B. The shafts 14 are rotatably disposed within two matching cylindrical passages 14A passing through the arms 12A and 12B of the framework 12. The U-shaped member 10 may be rotated around an axis 19 passing through the center of the shafts 14. The framework 12 is movably attached to two vertical beams 16A and 16B of an H-like member 16. The moving of the framework 12 along the beams 16A and 16B is accomplished by a first electrical motor 17 attached to the positioning device 1 and linked to the framework 12 by a gear-box (not shown), belt drive (not shown) and lead screws (not shown). The structure and use of the gear-box, belt drive and lead screws are well known in the art, are not the subject matter of the present invention and are therefore not disclosed in detail hereinafter. It will be appreciated by those skilled in the art that many alternative methods and devices for mechanically coupling a motor such as the motor 17 to the framework 12 which are well known in the art such as but not limited to rack and pinion mechanisms (not shown), various drive belts mechanisms, gear box mechanisms, helical screws and a variety of other suitable coupling mechanisms. Such alternative coupling mechanisms and devices may also be used in implementing the present invention. The framework 12 can be controllably moved along a direction perpendicular to the plane of the floor (not shown) such that the member 10 and the magnet assemblies 4 and 6 attached thereto are moved up and down with respect to the floor. The member 10 can be rotated in the directions represented by the arrows labeled 11 to vary the angle between the floor plane (not shown) and the arms 10A and 10B of the U-shaped member 10. The U-shaped member 10 can be rigidly fixed at a desired angle relative to the floor plane (not shown) by releasable locking mechanisms 15 for rigidly locking the shafts 14 within the arms 12A and 12B.

It is noted that, the positioning device 1 of FIG. 1 enables spatial positioning of the MRI probe 2 with three degrees of freedom including the vertical movement of the framework 12, the horizontal movement of the H-like member 16 and the rotation of the U-shaped member 10 around the axis 19. However, in accordance with other preferred embodiments of the present invention, the positioning device 1 may also be adapted to have additional degrees of freedom. For example, the U-shaped member 10 may be adapted to move horizontally along the axis 19 by adding a third motor (not shown) and a mechanical device such as a lead screw (not shown), a rack and pinion device (not shown), or another suitable coupling device or mechanism as disclosed hereinabove, for coupling the third motor to the U-shaped member 10 to provide controllable bidirectional horizontal movement of the U-shaped member 10 along the axis 19. Such a modification results in a positioning device capable of moving the MRI probe 2 with four degrees of freedom. Other suitable modifications of the positioning device 1 may also be implemented to add additional degrees of freedom to the moving of the MRI probe 2 by the positioning device 1.

In contrast to fixed magnet MRI systems known in the art in which the patient together with a motorized platform is moved into the bore of the magnet, preventing performing imaging during on-going surgery, the positioning device 1 of the present invention permits moving of the open MRI probe 2 with three degrees of freedom relative to the patient and allows precise positioning of the open magnetic probe 2 and the performing of imaging procedures during ongoing surgery without the need to move the patient as is disclosed in detail hereinafter.

Moreover, in contrast with MRI systems having a movable MRI probe, such as the system disclosed in U.S. Pat. No. 5,735,278 to Hoult et al., in which a large MRI probe is moved with two degrees of freedom relative to a patient fixed on an operating table, which do not permit imaging during ongoing surgery and require stopping of surgical procedures for imaging, the combination of the small sized open MRI probe 2 and the three degrees of freedom of the positioning device 1 of the present invention enables the precise positioning of the open MRI probe 2 relative to the organ or body part which is being imaged and allows imaging during on-going surgery obviating the need to move the MRI probe away from the imaged region for performing surgery or for simultaneously performing imaging and surgery.

It is further noted that, while the positioning device 1 of FIG. 1 is adapted for manual rotation of the U-shaped member 10 around the axis 19, other preferred embodiments of the present invention may be implemented in which the rotation of the U-shaped member 10 around the axis 19 is motorized. Such preferred embodiments may include a fourth motor (not shown) suitably coupled to the shafts 14 for controllably rotating the U-shaped member 10 around the axis 19.

It is still further noted that, the extent of the rotation of the U-shaped member 10 around the axis 19 may be mechanically limited by the beams 16A and 16B or by other parts of the positioning device 1. The H-like member 16 is movably attached to a base frame 20. The H-like member 16 can be moved horizontally along the base frame 20 by a second electrical motor 22 attached to the base frame 20 of the positioning device 1 and mechanically linked to a second gear- box, belt drive and lead screw (not shown) mechanism or to a rack and pinion mechanism (not shown), or any other suitable coupling mechanism disclosed hereinabove or known in the art The base frame 20 includes a docking member 24 for docking the positioning device 1 to an operating table (not shown in FIG. 1) as is disclosed in detail hereinafter.

The base frame 20 also includes wheels (not shown) or casters (not shown) rotatably attached thereto for facilitating the moving of the positioning device 1 on the floor in any selected direction. Any suitable type of wheels, casters and the like may be used to enable the rolling or moving of the positioning device 1 on a floor or other surfaces in any desired direction.

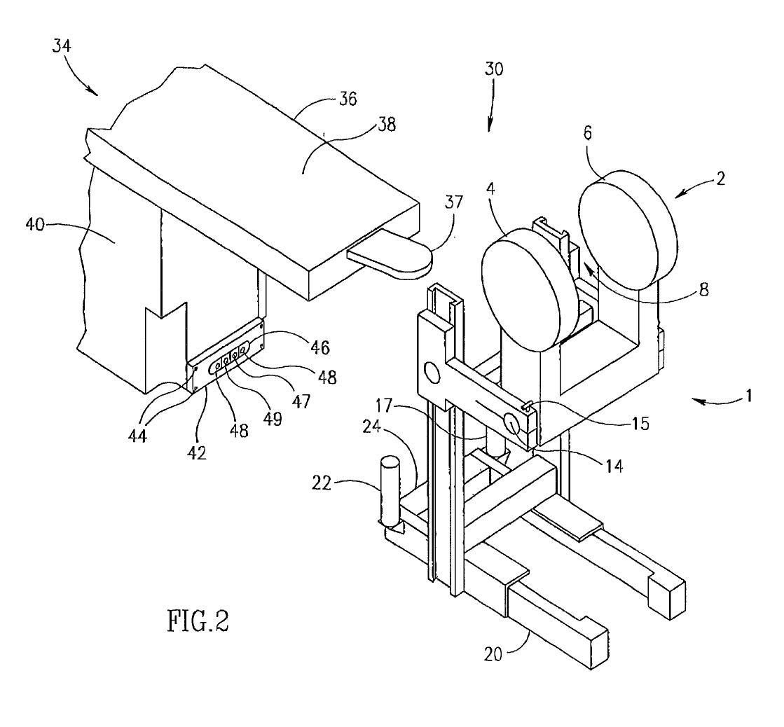

Reference is now made to FIG. 2 which is a schematic exploded view illustrating a system for interventional magnetic resonance imaging (iMRI) including an open magnetic probe, a positioning device and an operating table, in accordance with a preferred embodiment of the present invention. The system 30 includes the positioning device 1 and the open MRI probe 2 of FIG. 1. The system 30 further includes an operating table 34. The operating table 34 may be a standard operating table such as the “alphastar” operating table, commercially available from MAQUET Aktiengese(shaft, Germany. However, other types of commercially available operating tables may be adapted for use in the system 30.

The operating table 34 includes a patient supporting platform 36 having an upper surface 38 for supporting a patient (not shown) thereupon. The operating table 34 further includes a head holder 37 attached to the platform 36 for supporting the head of a supine patient (not shown) lying on the surface 38 of the platform 36. The head holder 37 is made from an electrically nonconducting non-ferromagnetic material such as plastic, fiberglass, or the like. The operating table 34 further includes a base 40 attached to the platform 36.

The operating table 34 further includes an adapter member 42 attached to the base 40. The adapter member 42 is a flat rectangular member made from steel or from any other suitable rigid material. The adapter member 42 is attached to the base 40 by screws 44 or by any other suitable means such as rivets or the like, The Adapter member 42 has a two holes 48 therewithin. The holes 48 are adapted to receive a plurality of docking pins (not shown) which protrude from the docking member 24 of the positioning device 1. The adapter member 42 may also include one or more locking devices (not shown) for locking the docking pins within the holes 48 to rigidly couple the positioning device 1 to the operating table 34.

The adapter member 42 may also include a connector panel 46 therein. The connector panel 46 includes an electrical connector 47 and a coolant connector 49. The electrical connector is connected by suitable electrical cables (not shown) passing under the floor of the operating room to an electronics cabinet (not shown) which includes gradient amplifiers (not shown), RF amplifiers (not shown), RF receivers (not shown), shim coil amplifiers, and other control units.

The electrical connector 47 is electrically connectable to a matched electrical connector (not shown) attached to the docking member 24. This matched electrical connector of the docking member 24 is electrically connected to the various electrical cables (not shown) which are connected to the MRI probe 2 for providing electrical power and appropriate signals to the various components (not shown) of the MRI probe 2. The electrical connector 47 provides the gradient coils (not shown) shimming coils (not shown), RF transmitting coils (not shown) of the MRI probe 2 of the with the appropriate electrical signals requires for operating the MRI probe 2 for imaging. The electrical connector 47 also provides an electrical path for feeding RF signals from the receiving RF coils (not shown) to the RF receiver circuitry (not shown). The electrical connector 47 also provides an electrical path for feeding control signals for controlling the operation of the motors 17 and 22 as is disclosed in detail hereinafter.

The coolant connectors 49 are sealingly connectable to matched connectors (not shown) attached to the docking member 24 for providing a coolant such as compressed air or water for cooling various parts of the MRI probe 2 such as the gradient coils (not shown) and the magnetic assemblies (not shown). Coolant carrying tubes (not shown for the sake of clarity of illustration) are attached to the coolant connectors 49 and pass under the floor of the operating room to a cooling system (not shown). This arrangement has the advantage of preventing any loose cables and coolant tubes from cluttering the floor area of the operating theater, freeing the floor area for unobstructed use by operating personnel.

It is noted that, while in the system 30 of FIG. 2 the positioning device 1 is rigidly and detachably attachable to the operating table 34, other embodiments of the system 30 may be constructed in which the positioning device 1 is not attachable to the operating table 34. Such systems (not shown) obviate the need for the pins and connectors attached to the docking member 24 and for the adapter member 42 of the operating table 34. Such systems may include other types of electrical cable connectors (not shown) and coolant tube connectors (not shown) which are well known in the art. In such systems, the positioning device (not shown) may be rigidly attached to the floor by bolts and nuts (not shown) or by other suitable locking mechanisms (not shown). Alternatively, the positioning device 1 may be wheeled to a position adjacent the operating table 34 and further movement thereof is prevented by using wheel brake mechanisms (not shown) for locking the wheels (not shown) of the positioning device 1.

Alternatively, in accordance with another preferred embodiment of the present invention the adapter member may be obviated and the electrical cables (not shown) and/or coolant carrying tubes (not shown) may be connected to the appropriate components of the open MRI probe 2 and simply laid on the floor of the operating room.

An advantage of the system 30 is that the positioning device 1 with the MRI probe 2 connected thereto are quickly movable between different locations such as storing locations and/or different operating rooms. When not in use, the positioning device 1 and the MRI probe 2 are stored in the operating room or in a storage room within a shielded storage cage (not shown). The positioning device 1 and the MRI probe 2 may be wheeled into the desired operating room and docked to the operating table 34 by using the docking member 42. After the operation is completed, the positioning device 1 and the MRI probe 2 connected thereto can be disconnected and detached from the operating table 34 and moved to the storage room or into another operating room where it is needed.

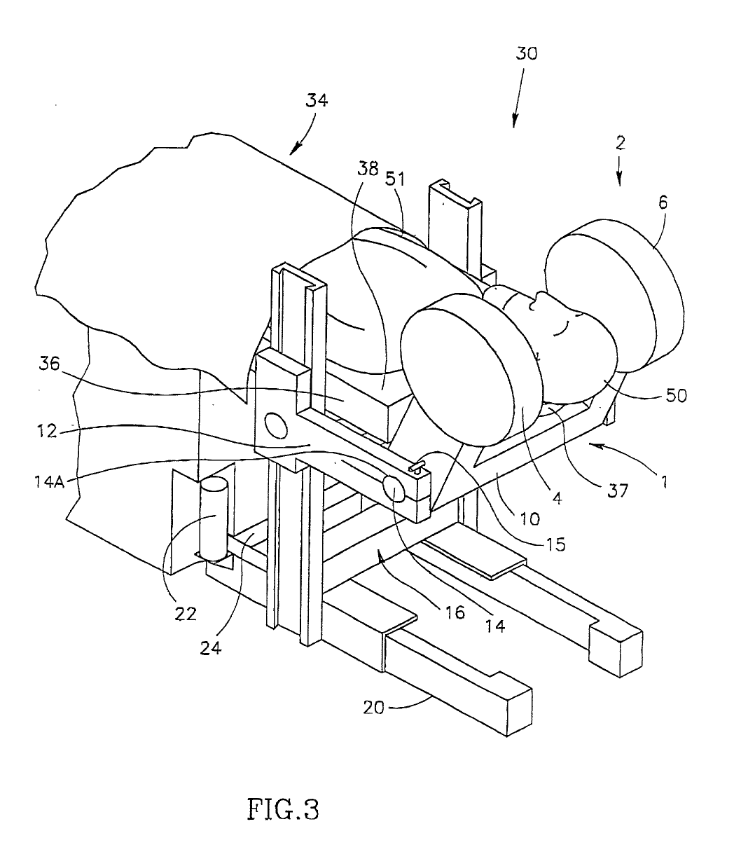

Reference is now made to FIG. 3 which is a schematic isometric view illustrating the iMRI system of FIG. 2, with the positioning device and MRI probe of FIG. 1 in an imaging position. In a typical iMRI situation, such as brain surgery, a patient 51 lies on the platform 36. The head 50 of the patient 51 rests on the head holder 37. Typically, the head 50 is attached to the head holder 37 by using a suitable attaching method and suitable attaching devices, such as but not limited to a stereotactic frame (not shown), suitable screws (not shown) which are attached to the head holder 37 and screwed into the skull of the patient, strips or belts (not shown) which fasten the head 50 to the head holder 37, or any other attaching or fastening method and/or devices which are known in the art. The attaching of the head 50 to the head holder 37 prevents movements of the head 60 during surgery and imaging and ensures that the MRI probe 2 may be repeatedly and accurately be positioned by the positioning device 1 at any selected allowed position for imaging.

When the head 50 is attached to the head holder 37, the various positions of the MRI probe 2 may be defined relative to the head 50 (or to any other body part which is attached to the operating table 34 for performing other types of surgery and imaging), or may be defined relative to the operating table 34 or any point thereof. The angle of the U-shaped member 10 relative to the floor plane (not shown) is adjusted to a desired value using a marker device (not shown in FIG. 3) as disclosed in detail hereinafter. The motors 17 and 22 are operated to position the framework 12 and the H-like member 16, respectively, such that the part of the head 50 to be imaged is-disposed within the field of view (FOV) of the open MRI probe 2. The imaged part of the head 50 of the patient 51 is thus disposed in the open region between the magnet assemblies 4 and 6. In this imaging position MRI can be performed before, during and after the surgical procedures. The open structure of the MRI probe 2 allows true interventional MRI in which MRI can be performed during surgery without moving the MRI probe 2 from it's imaging position.

If the surgical procedure requires the MRI probe 2 to be removed away from the region of the head 50 to improve access of the surgeon to the head 50 or for other purposes, the configuration of the positioning device 1 may be adjusted by operating the motors 17 and 22 to position the framework 12 and the H-like member 16, respectively, such that the MRI probe 2 and the positioning device 1 are retracted away from the region of the head 50 of the patient 51.

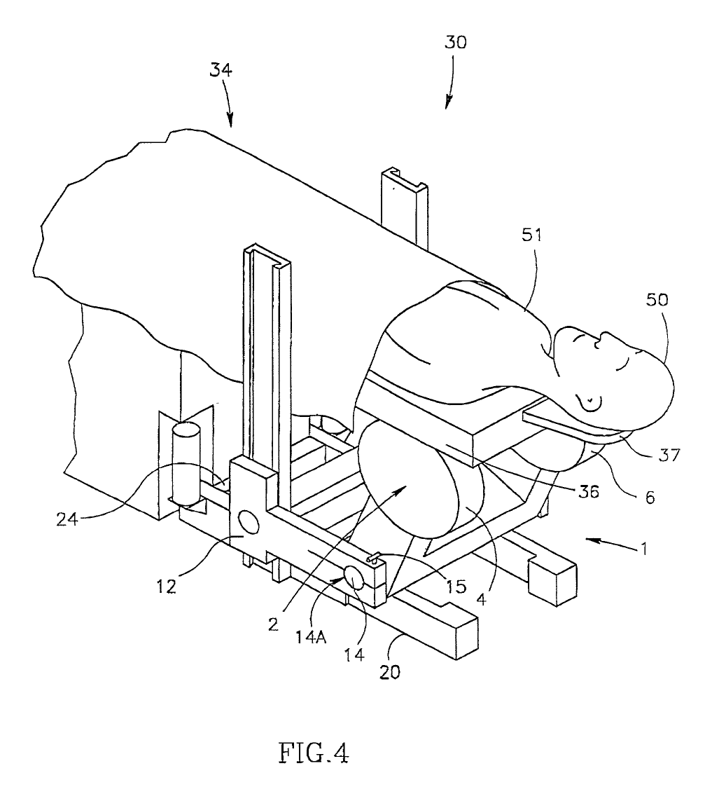

Reference is now made to FIG. 4 which is a schematic isometric view illustrating the iMRI system of FIG. 2, with the positioning device and the MRI probe of FIG. 1 in a retracted position under the operating table. In the retracted position, the MRI probe 2 and the framework 12 are disposed under the platform 36.

The advantage of the retracted position is the improved accessibility of the head 50. In the retracted position, the head 50 can be approached by a surgical instrument (not shown) or by other instruments (not shown) from directions that were not accessible in the imaging position illustrated in FIG. 3. The retracted position also has the advantage of providing improved visual access to the head 50. The removal of the magnet assemblies 4 and 6 under the platform 36 allows better visual inspection of regions of the head 50 which were visually obscured by the magnet assemblies 4 and 6 in the imaging position illustrated in FIG. 3. An additional advantage of the retracted position is that it allows the use near the operated organ or body part of equipment which is sensitive to strong magnetic fields. For example, when microscopes containing ferromagnetic metal parts need to be used during surgery for visual examination of the operated organ or for performing micro-surgery procedures under visual control, the MRI probe 2 and the positioning device 1 may be moved to the retracted position.

An advantage of the system 30 of FIGS. 2-4 is that the positioning device 1 and the MRI probe 2 can be wheeled into the operating room at the beginning of a procedure, and wheeled back-out when the procedure is completed. The operating room can therefore be used for all types of procedures, and does not need to be a dedicated iMRI operating room.

The open design of the MRI probe 2 allows the surgeon easy access to the operated organ so that surgery can be performed while the MRI probe 2 is in an imaging position. Thus, real-time or nearly real-time images can be obtained, permitting true interventional MRI.

When not required for a particular procedure, the positioning device 1 and MRI probe 2 can be detached and wheeled away from the operating table 34 so as to allow the use of ferromagnetic instruments in the operating room.

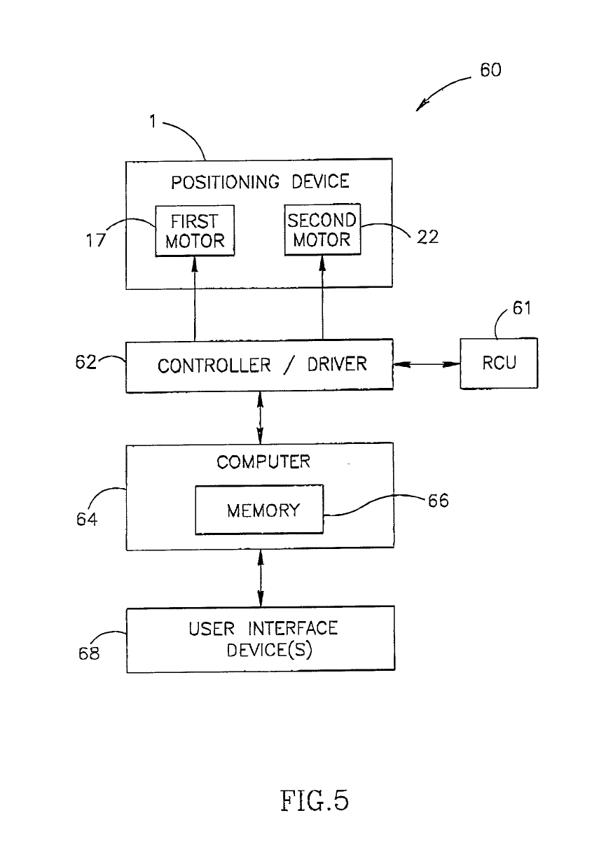

Reference is now made to FIG. 5 which is a schematic functional block diagram illustrating a position control sub-system for an iMRI system, in accordance with a preferred embodiment of the present invention. The position control sub-system 60 includes the positioning device 1 of FIG. 1 which includes the motors 17 and 22. The motors 17 and 22 are electrically connected to a controller driver 62.

The controller/driver 62 is a model SB 1003 motor controller, commercially available from AGS Electronic Ltd., Migdal Haemek, Israel. However, the controller/driver 62 may be any other suitable type of motor controller. A remote control unit (RCU) 61 is suitably connected to the controller/driver 62 for remote controlling of the moving of the MRI probe 2 by the user. The RCU 61 may be connected to the controller/driver 62 by a cable (not shown) or may communicate wirelessly with the controller/driver 62. The controller/driver 62 controls the operation of the motors 17 and 22 for moving the framework 12 and the H-like member 16, receptively, as disclosed hereinabove The controller/diver 62 is suitably connected to a computer 64 through an RS-232 interface or through any other suitable type of communication interface. The computer 64 may be a personal computer, a workstation, a mainframe or any other type of suitable computer. The computer 64 may also be the same workstation which is used for controlling the operation of the MRI system and for processing the acquired MRI signals and displaying the resulting MRI images. The computer 64 includes memory 66. The memory 66 may be any type of memory device suitable for data storage and retrieval. Preferably, the memory 64 is a random access memory (RAM) device. However, other types of memory or data storage devices may be used such as flash memory devices or a hard magnetic disk memory device or any other memory device or data storage device types known in the art.

The computer 64 is connected to a user interface device(s) 68. The user interface device 68 may be a keyboard (not shown), another RCU (not shown), a pointing device (not shown), a touch sensitive display screen (not shown) or any other device or combination of devices which may be used singly or in combination for providing input from the user to the computer and for providing output from the computer to the user. The user uses the user interface device(s) 68 for controlling the positioning of the MRI probe 2 and the positioning device 1 of FIG. 3. The computer 64 includes a control software program for manually or automatically controlling the moving of the positioning device 1 and for storing a plurality of positioning device position data in the memory 66.

During surgery, the operator may use the position control sub-system 60 to move the positioning device 1 and the MRI probe 2 into a position suitable for imaging of a selected portion of the head 50 (FIG. 3). For example, this may be achieved by using one or more of the user interface devices 68 connected to the computer 64 or the RCU 61 connected to the controller/driver 62 to provide numerical coordinate values or to input user commands representing incremental step-like movement instructions for the motors 17 and 22. However, other methods known in the art for manually or automatically controlling the movements of the motors 17 and 22 may also be used.

The operator may use the position control sub-system 60 to store a plurality of position data each representing a specific position of the positioning device. Each of the plurality of stored position data can be retrieved from the memory 66. This feature may be used in situations where the FOV of the MRI probe 2 is small and more than one imaging position is needed to provide images covering all the head regions of interest, or when the operated organ or body part is large and surgical procedures must be performed on different parts thereof which are separated by a distance which is larger than the longest dimension of the available FOV.

The operator may utilize each of the stored plurality of position data values by using a selected retrieved position data to control the controller/driver 62 for repositioning the positioning device 1 and the MRI probe 2 into a position corresponding to the position represented by the retrieved position data.

In a non-limiting example, the operator may use the user interface device(s) 68 to move the MRI probe 2 into a first position best for imaging a first brain region and to store a corresponding first position data after imaging of the first brain region. The operator may then use the user interface device(s) 68 to move the MRI probe 2 into a second position best for imaging a second brain region and to store the corresponding second position data after imaging of the second brain region. The operator may then use the user interface device(s) 68 or the RCU 61 to move the positioning device 1 and the MRI probe 2 into the retracted position under the platform 36 as illustrated in FIG. 3. While the MRI probe 2 is in the retracted position, surgery may be performed for opening the skull of the patient and performing other desired surgical procedures. The operator may then use the position control sub-system 60 to move the MRI probe 2 from the retracted position to the first position by using the retrieved position data of the first position. The operator may then acquire images of the first brain region while performing surgery on the first brain region. The operator may move the MRI probe 2 from the first position to the second position for imaging the second brain region while performing surgical procedures on the second brain region.

It is noted that, the operator may use the position control subsystem 60 to move the MRI probe 2 to a position which is not used for imaging and which is different from the fully retracted position illustrated in FIG. 4. For example, the operator may vertically lower the MRI probe 2 (FIG. 3) to a position in which the upper ends of the magnet assemblies 4 and 6 are positioned at a height equivalent to the height of the surface 38 of the platform 36. This position change improves accessibility of the head 50 during surgical procedures and also allows better visual examination of the head 50, and may be performed in less time than the time required to change into the fully retracted position illustrated in FIG. 3.

It is further noted that, the position control sub-system 60 keeps track of the current position of the MRI Probe 2 and includes pre-programmed data necessary to perform optimized moving of the MRI probe 2 between any two selected positions. The position control sub-system 60 also includes pre-programmed data for preventing moving of the MRI probe 2 in a “forbidden” trajectory which is not feasible mechanically or which may cause damage to the positioning device 1 or the patient 51. For example, when the MRI probe 2 is in the retracted position of FIG. 4, the control program will not enable the controller/driver 62 to activate the motor 17 to move the framework 12 in a vertical upward direction in a trajectory which will cause hitting of the underside of the platform 36 by the upper end of the magnet assemblies 4 and 6. Thus, the control program is adapted to use the preprogrammed data for allowing movement of the positioning device 1 only within an “allowed spatial envelope”. This allowed spatial envelope is determined, inter alia, by the dimensions of the MRI probe 2, the dimensions of the positioning device 1 and the dimensions of the operating table 34, and by their positions relative to each other. Such methods of movement optimization and limitation are well known in the art and will not be further disclosed herein.

It is noted that the position control sub-system 60 may be configured in a variety of different configurations. In accordance with one preferred embodiment of the present invention, the position control sub-system (not shown) includes the controller/driver 62 and the RCU 61. In this embodiment, the RCU 61 may be used by the user to control the position of the positioning device 1 and the MRI probe 2 as disclosed hereinabove but the controller/driver 62 is an autonomous controller/driver and is not connected to the computer 64.

In accordance with another preferred embodiment of the present invention, the position control sub-system (not shown) includes all the components of the position control sub-system 60. In this embodiment, the RCU 61 may be used by the user to control the position of the positioning device 1 and the MRI probe 2 as disclosed hereinabove and the controller/driver 62 is connected to the computer 64 for communicating its current position to the computer 64. However, in this embodiment the controller/driver 62 is autonomous and is not controlled by the computer 64.

It is further noted that since the FOV of small compact MRI probes may define a relatively small volume, it may be difficult to accurately position an MRI probe relative to the organ or body part which needs to be imaged for acquiring a good image covering the region of interest.

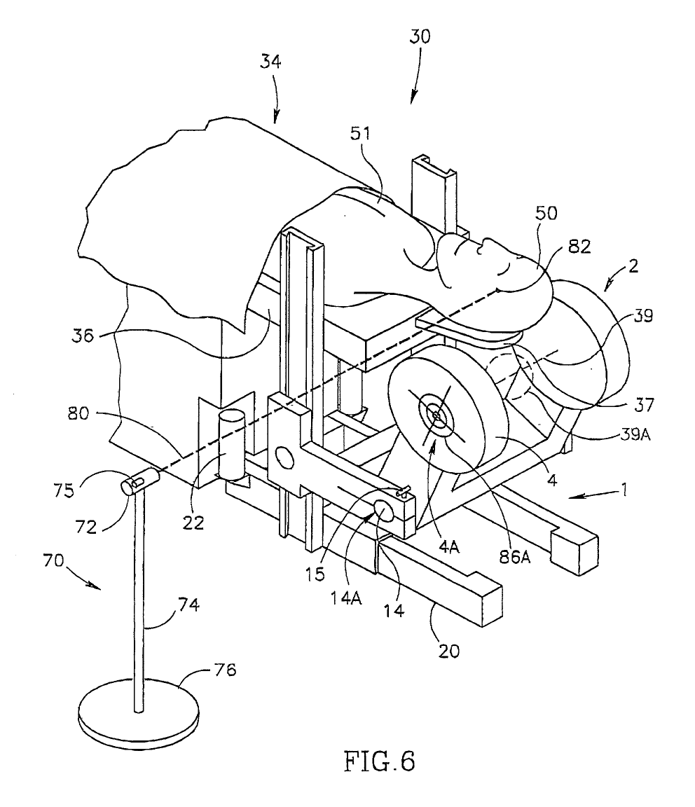

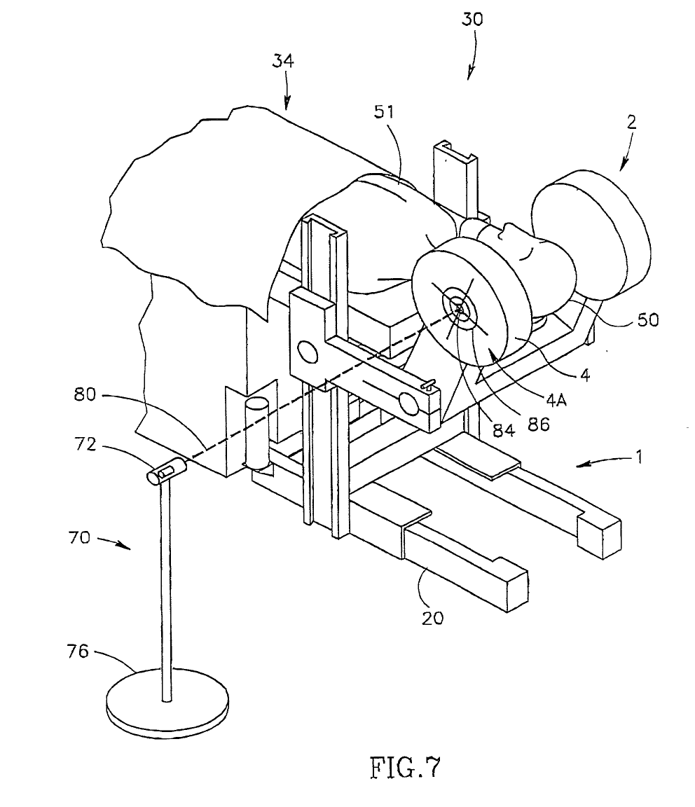

Reference is now made to FIGS. 6 and 7 which are schematic isometric views illustrating a marker device for positioning a magnetic probe in a desired imaging position, in accordance with a preferred embodiment of the present invention.

The marker device 70 of FIG. 6 includes a light source 72 attached to a supporting member 74. The supporting member 74 is attached to a base 76. The supporting member 74 is a variable length telescopically adjustable member (detail not shown) for adjusting the height of the light source 72 above the base 76. The light source 72 may be operated to produce a visible light beam 80. The light source may be any suitable visible light source capable of generating a collimated light beam. For example, the light source 72 may be a continuous wave laser or a pulsed laser such as a diode laser or any other type of suitable laser. The light source 72 may also be a source of incoherent light coupled to collimating optics (not shown) The source of incoherent light may be a light bulb such as a quartz-halogen light bulb, a tungsten filament light bulb or any other suitable type of light bulb. The light source 72 may be suitably connected to an electrical power outlet (not shown). Alternatively, a power source (not shown) may supply the power for operating the light source 72. The power source may be an external power source such as an AG/DC transforming adapter (not shown) or an internal power source such as batteries (not shown) contained within the light source 72. The light source 72 also includes a switch 75 for switching the light source 72 on and off.

The marker device 70 of FIG. 6 may be used for positioning the MRI probe 2 of the iMRI system 30. After the patient 51 is placed on the platform 36 of the operating table 34 and the head of the patient 50 rests on the head holder 37, the positioning device 1 is moved using the position control sub-system 60 disclosed hereinabove such that the MRI probe 2 is positioned under the head holder 37. The operator then positions the marker device 70 near the positioning device 1 and switches the light source 72 on to direct a light beam 80 onto the head 50. The operator then moves the marker device 70 to roughly direct the light beam 80 at the head 50 and to align the light beam 80 so that it is approximately perpendicular to the plane of the outward facing surface 4A of the magnet assembly 4. This alignment is a rough visually performed adjustment. The operator then further adjusts the position of the light source 72 by using the adjustable supporting member 74 and by moving the marker device 70 such that a light spot 82 is projected by the light beam 80 on the head 50. The light spot 82 is positioned on the head 50 such that the path of the light beam 80 will approximately coincide with the horizontal axis 39 passing through the center of the imaging volume 39A when the MRI probe 2 is re-positioned such that it's FOV is placed in the desired position for imaging a particular part of the brain (not shown).

Turning to FIG. 7, the operator then moves the positioning device 1 and the MRI probe 2 attached thereto, using the position control sub-system 60 disclosed hereinabove, to a position in which the light spot 84 projected by the light beam 80 on the surface 4A coincides with a cross-hair shaped marker 86 marked on the surface 4A. The crossing-point of the cross-hair marker also lies on the imaginary horizontal axis (not shown) of the FOV of the MRI probe 2. If desired, the operator may then proceed to perform imaging to verify the correct placement of the FOV then store the position of the positioning device 1 as position data as disclosed in detail hereinabove- The operator may also proceed to repeat the above disclosed procedure for marking and storing other desired imaging. positions. After the desired position data has been stored, the operator may start surgical procedures and perform iMRI as disclosed hereinabove.

It is noted that, while the marker 86 is a cross-hair marker, other types of markers may be used such as a circle shaped marker, a dot shaped marker or any other suitable type of marker. Preferably, the light beam 80 is such that the diameter of the projected light spot 82 is between 0.2-0.5 centimeter. However, other spot diameters may also be used.

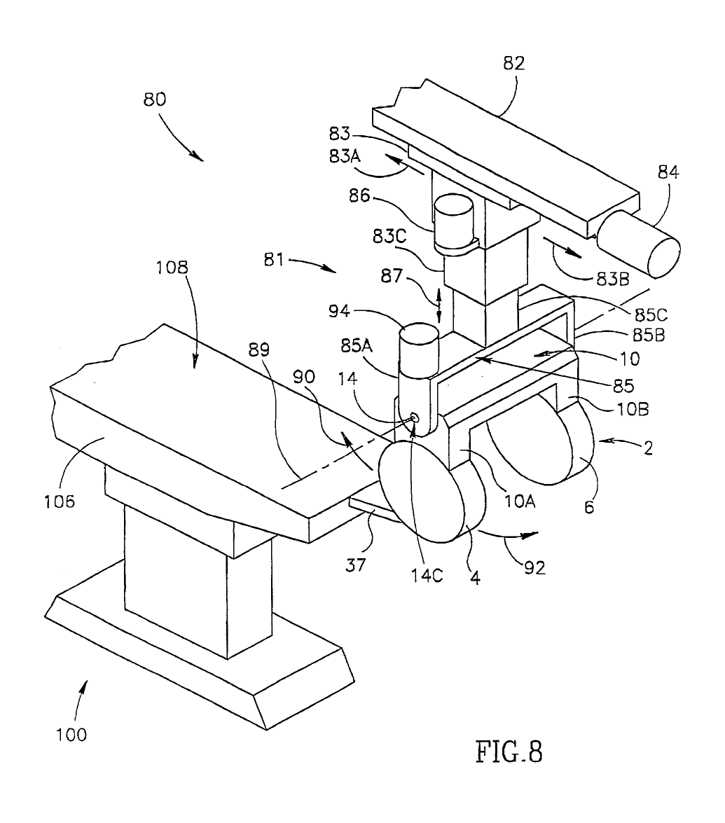

Reference is now made to FIG. 8 which is a schematic isometric view illustrating an iMRI system having a ceiling-mounted positioning device for intra-operative positioning of an open MRI probe, in accordance with another preferred embodiment of the present invention. The system 80 includes a positioning device 81, an open MRI probe 2 and an operating table 100. The operating table 100 has a patient supporting platform 106 having an upper surface 108. The MRI probe 2 is rotatably attached to the positioning device 81 by two shafts 14 attached to a U-shaped member 10. The positioning device 81 includes an elongated track member 82. The track member 82 is rigidly attached to the ceiling (not shown) of an operating room. The positioning device 81 further includes a base member 83. The base member 83 is movably attached to the track member 82 and may be bidirectionally moved therealong in the directions indicated by the arrows 83A and 83B by a motor 84 attached at an end of the track member 82. The base member 83 has a hollow portion 83C. The positioning device 81 includes a three-armed member 85 having an upper arm 85C and two lower arms 85A and 85B. The upper arm 85C of the member 85 is movably attached within the hollow portion 83C of the base member 83. The arm 85C of the member 85 is movably attached within the hollow portion 830 and is movable within the hollow portion 830 by a motor 86. The motor 86 is attached to the base member 83 and is suitably mechanically coupled to the arm 85C. The entire member 85 is bidirectionally movable in the directions represented by the double headed arrow 87.

The positioning device 81 further includes an open MRI probe 2 similar to the MRI probe 2 of FIG. 1. The two arms 10A and 10B of the U-shaped member 10 of the MRI probe 2 are attached to the two magnetic assemblies 4 and 6, respectively. The U-shaped member 10 is rotatably attached to the member 85 by two shafts 14 which are rigidly attached to the U-shaped member. The shafts 14 are rotatably disposed within two matching cylindrical passages 14C passing through the arms 85A and 85B of the member 85. The U-shaped member 10 may be rotated around an axis 89 passing through the center of the shafts 14 in the directions indicated by the arrows 90 and 92. The rotation of the U-shaped member 10 is performed by a motor 94 attached to the member 85. The shaft (not shown) of the motor 94 is rotatingly coupled to one or more of the shafts 14 disposed within the arm 85A by a suitable coupling mechanism, such as but not limited to, a gear box (not shown, cogwheels (not shown), a rack and pinion mechanism (not shown), a suitable drive belt, a worm wheel or by any other suitable coupling mechanism known in the art.

The positioning device 81 of the iMRI system 80 can spatially move the MRI probe 2 with three degrees of freedom. The operating table 100 has a head holder 37 attached to one end thereof.

The positioning device 81 can move the MRI probe 2 into a plurality of positions relative to the operating table 100. In some of these positions the MRI probe 2 is withdrawn away from the operating table 100 and the patient (not shown) lying thereupon. In other positions of the positioning device 81 the MRI probe 2 is positioned such that its FOV (not shown) at least partially coincides with the organ or body part to be imaged (not shown). The latter positions are referred to as imaging positions.

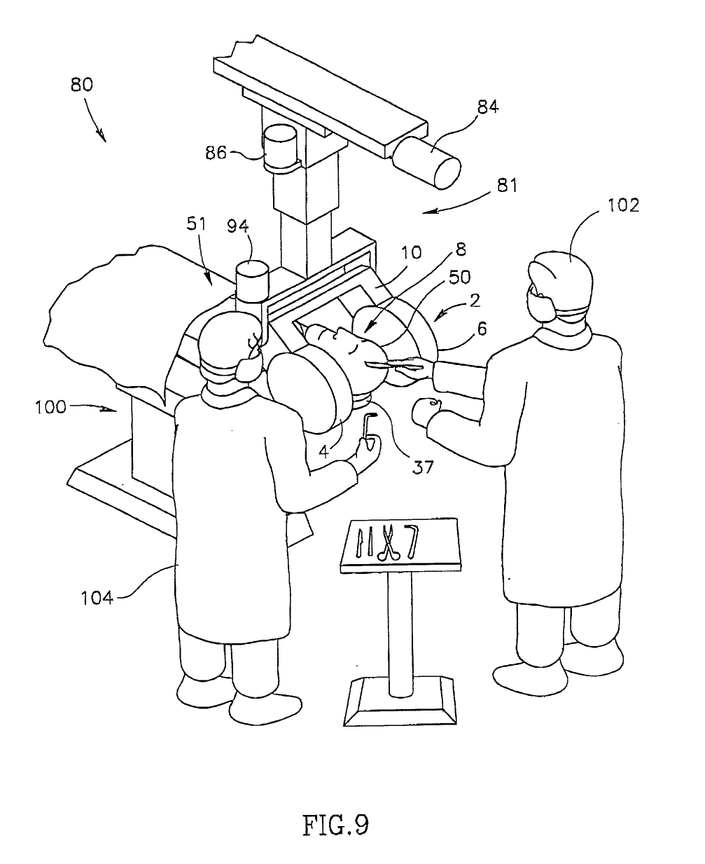

Reference is now made to FIG. 9 which is a schematic isometric view illustrating the iMRI system of FIG. 8, With the MRI probe in an imaging position.

FIG. 9 illustrates the MRI probe 2 positioned such that the head 50 of a patient 51 is placed in the open region 8 between the two magnet assemblies 4 and 6. This positioning is achieved by suitably controlling all or some of the motors 84, 86 and 94. The positioning of the MRI probe 2 by the positioning device 81 is controlled by a position control sub-system similar to the position control sub-system 60 of FIG. 5, except that the positioning device 81 has three motors 84, 86 and 94 and that the controller/driver (not shown) is a controller driver capable of controlling and driving the three motors 84, 86 and 94. The positioning device 81 may be controlled and positioned relative to the operating table 100 as disclosed in detail hereinabove for the position control sub-system 60, except that the different positions are different from the positions possible for the positioning device 1. Due to the dissimilar spatial relation and orientation of the MRI probe, the operating table and the positioning device relative to each other. The position control sub-system (not shown) of the system 80 may be configured in all of the configurations disclosed in detail hereinabove for the position control subsystem 60 of FIG. 5, including autonomous operation of the controller/driver for positioning the MRI probe 2, computer monitored positioning of the MRI probe 2 by an autonomous controller/driver, and full computer control of the controller/driver with automatic position storage and automatic computer repositioning of the MRI probe 2. Additionally, the allowed spatial envelope of the system 80 will be different than the allowed spatial envelope of the system 30 due to the different construction and dimensions of the positioning device and possibly of the operating table of the systems 80 and 30.

In operation, the patient 51 is positioned on the operating table 100 with the head 50 resting on the head holder 37. The operator of the system 80 may position the MRI probe 2 at a retracted position away from the patient 51. This may be done before during and after the operation to allow easy access to the patient by the surgeon 102 and by other medical or technical users such as the nurse 104. The retracted position is also useful for permitting the use of surgical or other equipment that cannot be operated near a strong magnetic field, as disclosed in detail hereinabove.

Before and during the operation, the operator of the system 80 may position the MRI probe 2 at an imaging position for obtaining MRI images of the operated organ or body part. Similar to the system 30, the system 80 has the advantage that the MRI probe 2 allows relatively unhindered access of the operated region by the surgeon 102 and that if a higher degree of accessibility is required, the MRI probe 2 can be quickly and automatically lifted upwards or moved aside or repositioned into a selected or pre-selected one of the retracted positions disclosed hereinabove. For some operational procedures, the MRI probe 2 may be left in an imaging position for the entire duration of the operational procedure and MRI images may be acquired at any desired time during the operation.

An additional advantage of the system 80 is that the mounting of the positioning device 81 on the ceiling of the operating room improves even further the accessibility of the patient 51 and the operating table 100, since most parts of the positioning device 81 are not positioned close to the operating table 100 or to the patient 51. Typically, during the operation the area surrounding the operating table is not obstructed by the positioning device 80 and is relatively freely accessible.

The positioning of the MRI probe 2 in an imaging position may be assisted by the marker device 70 of FIGS. 6 and 7, using a method similar to the method of positioning disclosed in detail hereinabove and illustrated in FIGS. 6-7 taking into account the structural differences between the system 30 and the system 80.

It is noted that, while the operating tables 34 and 100 of FIGS. 2 and 8 respectively, are illustrated with their upper surfaces 38 and 108 respectively being flat and parallel to the floor of the operating room, many other possible configurations of the operating tables 34 and 100 are possible which are within the scope of the present invention.

For example, the patient supporting platforms 36 and 106 may be tilted at an angle (not shown) to the floor of the operating room. Moreover, the patient supporting platforms 36 and 106 may comprise multiple platform parts (not shown) each of which can be moved or tilted with respect to the other parts. In a non-limiting example, the operating table 34 referred to hereinabove has a patient supporting platform which may be tilted in any direction (i.e has roll and pitch capabilities) with respect to the base of the operating table or to the floor upon which the operating table stands. Alternatively or additionally, the operating table 34 may include two parts of the platform (not shown) which may be moved or tilted with respect to a third central platform part (not shown) attached between the two outermost platform parts. Other commercially available operating tables may have other different part configurations which may allow other types of movements and different shapes of their patient supporting platforms.

In some types of brain surgery, the surgeon may prefer to configure the operating table 34 or 100 in a chair-like configuration as is known in the art. In such a configuration, the patient is positioned in the operating table in a sitting like position. The positioning devices 1 and 81 of the present invention are adapted to allow such different configurations of the operating tables 36 and 100, respectively.

Therefore, the position control sub-system 60 of FIG. 5 may be adapted to operate in two different modes. In a first operating mode, the various positions and the position coordinate data of the MRI probe 2 are defined relative to the operating table 36 (FIG. 2) or 100 (FIG. 8). This operating mode is useful in situations in which the operating table 36 has a single fixed configuration and does not have any moving or tiltable parts and is rigidly attached to the positioning device 1. This operating mode is also useful in situations in which the operating table 100 has a single fixed configuration and does not have any moving or tiltable parts and is rigidly attached to the floor of the operating room while to the positioning device 81 is attached to the ceiling of the operating room, thus providing a common “reference frame” for defining the position of the MRI probe 2 relative to the operating table 100.

However, this first mode may also be used with operating tables having moving and/or tiltable parts as long as the operating table has at least one part which is fixed and does not move. In this alternative, if preprogrammed data for allowing movement of the positioning device only within the allowed spatial envelope as disclosed hereinabove, the preprogrammed data has to be adapted such that it takes into account all the possible positions of all the relevant movable parts of the operating table in order to properly define the allowed trajectories.

In the second operating mode of the position control sub-system 60, the various positions and the position coordinate data of the MRI probe 2 are defined relative to a point on or within the body part to be imaged. In this mode, after the MRI probe 2 has been positioned in a selected imaging position as disclosed in detail hereinabove, this selected position may be defined as a reference position and all the other MRI probe positions are then defined relative to the reference position and appropriately stored. In this second operating mode, if preprogrammed data is used for allowing movement of the positioning device only within the allowed spatial envelope as disclosed hereinabove, the coordinates of the position of the operating table will be included in the preprogrammed data based on the known position and dimensions of the operating table which for a stationary non-configurable operating table are fixed with respect to the operating room reference frame. Alternatively, if the operating table is of the configurable type having movable and tiltable parts as disclosed hereinabove, the preprogrammed data for allowing movement of the positioning device only within the allowed spatial envelope as disclosed hereinabove, has to be adapted such that it takes into account all the possible positions of all the relevant movable parts of the operating table in order to properly define the allowed trajectories.

Both of the operating modes of the position control sub-system 60 of FIG. 5 may be used with systems in which the positioning device 1 is rigidly attached to the operating table 36 as disclosed hereinabove, and the positioning device 81 is attached to the ceiling of the operating room as disclosed hereinabove. Alternatively, in accordance with other preferred embodiments of the present invention, the operating modes of the position control sub-system 60 of FIG. 5 may also be used with the system 30 in which the positioning device 1 is not attached to the operating table 36. In such cases, the coordinates of the MRI probe 2 and/or the positioning device 1 and or both sets of position coordinates have to be manually entered as data into tho position control sub-system 60 in order to properly define the allowed trajectories as disclosed hereinabove.

These position coordinates or position data may be obtained using various methods. For example, after the positioning device 36 is placed in the desired position for performance of iMRI procedures, the positioning device is rigidly attached to the floor of the operating room by using any of the locking devices such as the nuts and bolts or the lockable wheel brakes disclosed hereinabove to fixate the position of the non moving base frame 20 of the positioning device 1 relative to the floor of the operating room. The distance between known reference points (not shown) on the operating table 36 and known reference points (not shown) on the positioning device 1 is determined and provided as input to the position control sub-system 60 of FIG. 5. This data together with the preprogrammed data of the dimensions of the operating table 36 and the positioning device 1 (including the dimensions of the MRI probe 2 attached to the positioning device 1) and of all the possible dimensions of all the possible configurations which the operating table 36 and the positioning device 1 may assume, is used to compute the allowable trajectories for moving the MRI probe 2. Standard computational methods may be used for calculating such allowable trajectories and for computing the control signals required for controlling the moving of the various parts of the positioning devices 1 and 81 to move the MRI probe 2 between any selected position and any other selected position.

Alternatively, the distance between the reference points of the operating table and the positioning device 1 may be automatically determined and communicated to the position control sub-system 60 of FIG. 5. after fixation of the positioning device 1 to the floor of the operating room. The distance may be determined by any suitable range finding methods (having sufficient accuracy) known in the art, such as but not limited to laser beam range finding methods, ultrasound acoustic range finding methods or any other methods for accurate determination of object coordinates in a reference frame which are known in the art, including automated and manual measuring methods. The automatically determined distances between the reference points may be manually or automatically communicated to the computer 64 of the sub-system 60, using any of the interface devices 68 of FIG. 5 or by a suitable communication line (not shown) suitably interfaced to the computer 64, or by wireless communication methods, such as but not limited to, radio frequency telemetry, infra-red based telemetry and the like which are known in the art of telemetry and remote wireless communication.

An advantage of the above automatic determination and communication methods is that they do not involve any manual position measuring steps, thus reducing the possibility of human operator errors.

It will be appreciated by those skilled in the art, that while the positioning devices 1 and 81 of the present application are preferably implemented such the the MRI probe 2 may be moved with three degrees of freedom as disclosed hereinabove, in accordance with other different embodiments of the present invention, the positioning device may be adapted to move the MRI probe 2 with a smaller or greater number of degrees of freedom. For example, in accordance with one preferred embodiment of the present invention, the device 1 of FIG. 1 is adapted to operate with only one degree of freedom by fixing the framework 12 to the vertical beams 16A and 16B of the H-like member 16, such that the framework 12 is not free to move vertically, and by fixedly attaching the U-shaped member 10 to the framework 12. In such an embodiment the need for the motor 17(FIG. 1) is obviated and the motor 17 is therefore not included in the device. In this preferred embodiment the MRI probe 2 may be moved only horizontally using the motor 22 for moving the H-like member 16 with respect to the base frame 20.

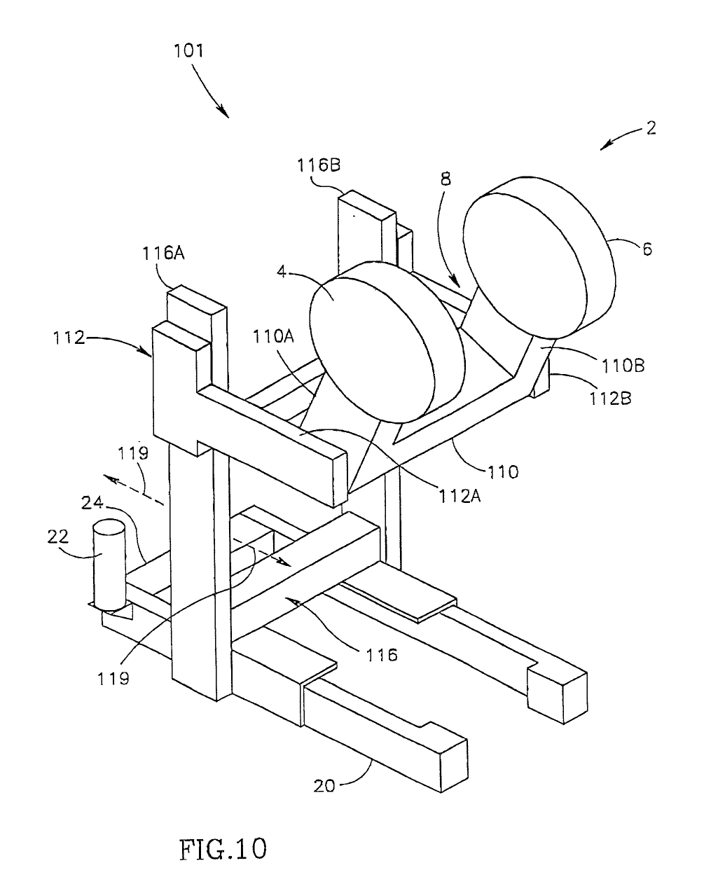

Reference is now briefly made to FIG. 10 which is a schematic isometric view of a positioning device 101 for intra-operative positioning of an open MRI probe in an iMRI system, adapted for moving the MRI probe with one degree of freedom, in accordance with another preferred embodiment of the present invention.

The device 101 has some parts which are identical to the corresponding parts of the positioning device 1 of FIG. 1 (these parts are labeled by identical reference numerals), except that the H-like member 116, the framework 112 and the U-shaped member 110 of the positioning device 101 are different than the H-like member 16, the framework 12 and the U-shaped member 10, respectively, of the positioning device 1 of FIG. 1 and that the motor 17 of FIG. 1 and the mechanical coupling means (not shown in FIG. 1) which operatively couple the motor 17 to the framework 12 of FIG. 1 are not included in the positioning device 101 of FIG. 10.

The framework 112 has two arms 112A and 112B which are rigidly attached to the arms 110A and 110B of the U-shaped member 110 by bolts and nuts (not shown) or by welding (not shown) or by any other suitable attachment method known in the art. The U-shaped member 110 is tilted at a fixed angle with respect to the horizontal plane (not shown) on which the base frame 20 is supported (which plane is approximately parallel to the floor of the operating room). The arms 112A and 112B of the framework 112 are rigidly attached to the vertical beams 116A and 116B, respectively, of the H-like member 116. The motor 22 of the positioning device 101 is movably coupled to the H-like member 116 such that the H-like member 116, the framework 112, the U-shaped member 110 and the magnet assemblies 4 and 6 of the MRI probe 2 may all be moved as a single unit with respect to the base frame 20 in the directions indicated by the arrows 119. This arrangement of the device 101 allows for the controlled positioning of the MRI probe 2 in a plurality of different positions using movements with a single degree of freedom. The positioning device 101 may be attached to an operating table (not shown) using any of the methods and devices for attachment as disclosed hereinabove in detail for the positioning device 1 of FIG. 1. Thus for the performing iMRI procedures including but not limited to imaging and surgery, the MRI probe 2 may be moved into a first imaging position relative to the body part or organ of the patient. The first imaging position enables the performing of imaging procedures and surgery procedures without having to move the MRI probe 2 from the first imaging position.

The MRI probe 2 may also be moved into other different positions which may be stored as position data as disclosed hereinabove for the positioning device 1 of FIG. 1, with the exception that all these positions are reached by moving the H-like member 116 along a straight line (not shown) parallel to the arrows 119 and that the controller 62 of FIG. 5 now controls only a single motor ( which is the motor 22 of FIG. 10) for moving the H-like member 116 of the positioning device 101. Thus, if the surgeons need better access to the organ or body part undergoing surgery than the access allowed when the MRI probe 2 is in the first imaging position, the MRI probe 2 may be moved into a retracted position which is at a distance from the organ or body part. The MRI probe 2 may later be moved back into any other selected allowable position including but not limited to the first imaging position, either manually by using any of the user interfaces 68 or the RCU 61 of FIG. 5, or automatically by specifying a position for which data was stored in the memory 66 of the computer 64 (FIG. 5).

Other preferred embodiments of the positioning devices 1, 81 and 101 of the present invention may be constructed by suitably increasing or decreasing the number of degrees of freedom of the moving of the MRI probe by the positioning device 1 or 81 by appropriately modifying one or more of the various movable parts of the devices 1 81 and 101 such that they are fixedly attached to the appropriate parts of the positioning device 1 or 81, or by adding additional parts thereto that provide additional translational or rotational degrees of freedom to the movements of the MRI probe 2. The preferred embodiments of the positioning devices 1 and 81 of the present invention, having less than 3 degrees of freedom of movement have the advantages of greater simplicity and reduced cost of construction at the cost of limiting the choice of the possible movements that can be performed by the devices. However, such simpler embodiments having two or one degrees of freedom may be fully adequate for some type of iMRI while being less expensive to construct and being easier to operate and maintain. Thus, as long as any of the embodiments of positioning devices disclosed hereinabove has the capability to move the MRI probe 2 from any selected position into at least one position which enables the performance of both surgical and imaging procedures, either simultaneously or sequentially, on a body part or organ of a patient without having to move the MRI probe 2 from that position, such an embodiment has true iMRI capabilities and is considered within the scope of the present invention. In such a position of the positioning device of the present invention, surgical procedures may be followed by imaging procedures without changing the position the MRI probe. Alternatively, such a position may be used for performing MRI imaging of the body part which is undergoing surgery during the performing of the surgical procedures without having to interrupt the surgical procedures and without having to change the position of the MRI probe from this position. It is noted that more than one such position may exist depending. inter alia, on the precise construction, dimensions and implementation of the positioning device 1, the MRI probe 2 and on the size and dimensions of the organ or body part which is undergoing surgery or other therapeutic procedures.

It will be appreciated by those skilled in the art that while in the preferred embodiments of the invention disclosed hereinabove, the positioning device for positioning the open magnetic probe is shown attached to the floor or to the ceiling of an operating room, in accordance with other preferred embodiments of the present invention the positioning device (not shown) may be attached to one or more of the walls of the operating room, Additionally the positioning device of the present invention may also be attached to a wall and to the ceiling of the operating room or to any suitable combination selected from a wall, the floor and the ceiling of the operating room or of the room within which the magnetic probe of the invention is disposed.

It is further noted that, while the iMRI system of the present invention is adapted to include open MRI probes which are generally C-shaped, U-shaped or Y-shaped as disclosed hereinabove, other preferred embodiments of the invention may be adapted for use with other shapes of open MRI probes which are suitable for performing iMRI.

It is yet further noted that, while the iMRI system of the present invention is adapted to perform iMRI on the brain, other preferred embodiments may be adapted for performing iMRI on other organs or body parts of a patient such as a knee or the back of a patient or any other organ or body part which can be accommodated within the open region 8 of the MRI probe 2. Such embodiments are considered to be within the scope of the present invention.

It is still further noted that, while the preferred embodiment of the positioning device 1 disclosed hereinabove includes two motors, other preferred embodiments may include only one motor (not shown) which is controllably and selectively couplable to the framework 12 or the H-like member 16 (FIG. 1). In such an arrangement, the single motor may be controllably coupled to a selected one of the framework 12 and the H-like member 16. This arrangement allows for using a single motor for performing the movements performed by the motors 17 and 22 of the positioning device 1 of FIG. 1. However, in the single motor embodiment, the framework 12 and the H-like member 16 must be moved sequentially and cannot be moved simultaneously as is possible in the positioning device 1 of FIG. 1.

Similarly, the positioning device 81 of the system 80 may include a number of motors which is smaller or larger than three. Additionally, some of the three or more degrees of freedom of moving the MRI probe 2 of the system 80 may also be achieved by any non-motorized manual means known in the art.

It is still further noted that, the iMRI systems 30 and 80 (FIGS. 3 and 9, respectively) of the present invention include all the necessary components for performing MRI. Some of these components, such as shimming coils (not shown), gradient coils (not shown), transmitting RF coil(s) (not shown), are included within the MRI probe 2. Other components such as receiving RF coil(s) (not shown), are placed on the patient's head or on another imaged organ or body part. Additional components (not shown) of the system 30 include a temperature control system (not shown), and the rest of the electronic circuitry used for operating the various coils, for controlling the system and for processing and displaying the images. The construction and operation of all these components is well known in the art and is not the subject matter of the present invention.

It will be appreciated by those skilled in the art, that while typically, the open MRI probe 2 includes the components necessary for magnetic resonance imaging such as, but not limited to internal or external gradient coils or a combination thereof, shimming coils, receiving RF coils, transmitting RF coils, temperature control elements for stabilizing the temperature of the magnet assemblies 2 and 4, and the like, some of the components need not be integrated within the open MRI probe 2. For example, the receiving RF coils, or the receiving/transmitting RF coils may be separate coils which are adapted for a specific organ or body part. Such RF coils may be positioned on or near the organ or body part to be imaged without being attached to the MRI probe 2. Thus, in such a case, when the MRI probe 2 is moved from one position to another position, the receiving RF coil (not shown) or the receiving/transmitting RF coil(s) is not moved and stays at the same position relative to the body part or organ which is to be imaged.

Therefore throughout the present application and the following claims, the term “open MRI probe” is not limited to denoting an open MRI probe which includes magnetic assemblies for creating a region of homogenous magnetic field suitable for performing magnetic resonance imaging and all of the other components, such as gradient coils, RF coils, and shimming coils which are necessary for performing magnetic resonance imaging. Rather, the term open MRI probe may also denote an open magnetic probe which includes magnetic assemblies but does not necessarily structurally include all of the other components necessary for obtaining an MRI image. For example, the open MRI probe may include magnetic assemblies having permanent magnets, internal or external gradient coils, but does not include an RF coil. In accordance with another non-limiting example, the open magnetic probe may include magnetic assemblies having permanent magnets, internal or external gradient coils, and an active shimming coil but no RF coil. In accordance with yet another non-limiting example, the open magnetic probe may include magnetic assemblies having permanent magnets, internal or external gradient coils, an active shimming coil and an RF coil. Many other such permutations and modifications of the components included in the open MRI probe are thus possible depending, inter alia, on the structure, dimensions and design of the open magnetic probe, on the type of the magnets providing. the main magnetic field of the MRI probe (i.e permanent magnets, resistive electromagnets, superconducting electromagnets, and any combination thereof, the degree of main field homogeneity required for the particular imaging application, and the imaged organ or body part.

It is yet further noted that while the motors 17, 22, 84, 86 and 94 are preferably electrical motors, other types of suitable motors known in the art may also be used.

It is further noted that the framework 12, the H-like member 16 and the base frame 20 of the positioning device 1 (FIG. 1) collectively define a movable framework, throughout the specification and the claims of the present application. Similarly, the framework 112, the H-like member 116 and the base frame 20 of the positioning device 101 (FIG. 10) also collectively define a movable framework, throughout the specification and the claims of the present application. Finally, the track member 82, the base member 83, the three armed member 85 of the positioning device 81 (FIG. 8) also collectively define a movable framework, throughout the specification and the claims of the present application. It will be appreciated by those skilled in the mechanical art, that the various members of movable frameworks defined hereinabove may be modified and changed in many ways which are all regarded to be within the scope and spirit of the present invention. Therefore, the particular construction of and the specific parts included in any of the movable frameworks illustrated in FIGS. 1, 8 and 10 are given by way of illustrative examples only and are not intended to limit the scope of the invention. Thus, the number, shape and connectivity of the various components or parts of the different movable frameworks illustrated in FIGS. 1,8 and 10 may be changed and modified at will as long as the resulting movable framework is adapted for controllably moving an open MRI probe attached thereto between the probe positions as is disclosed in detail hereinabove.

While the invention has been described with respect to a limited number of embodiments, it will be appreciated that many variations, modifications and other applications of the invention may be made.

What is claimed is:

1. A system for magnetic resonance imaging, the system comprising:
an operating table for supporting a patient;
a magnetic resonance imaging system including an open magnetic probe for producing a volume of substantially homogenous magnetic field for imaging a body part of said patient; and
a controllable positioning device attached to said open magnetic probe for controllably positioning said open magnetic probe in a position selected from a plurality of probe positions, said plurality of positions includes at least one imaging position in which said open magnetic probe is positioned relative to said body part of said patient such that surgical procedures and imaging procedures can be performed on said body part of said patient while said open magnetic probe is positioned in said at least one imaging position.

2. The system according to claim 1 wherein said positioning device comprises a controllably movable framework attached to said open magnetic probe and a position control unit operatively connected to said movable framework for controlling the moving of said open magnetic probe by said movable framework from one position of said plurality of probe positions to another position of said plurality of probe positions.

3. The system according to claim 2 wherein said movable framework is a motorized framework comprising at least one motor operatively coupled to said framework for moving said magnetic probe relative to said operating table with at least one degree of freedom.

4. The system according to claim 2 wherein said position control unit comprises a microprocessor in communication with a data storage unit for retrievably storing data representative of said plurality of probe positions and for controlling the moving of said magnetic probe into a probe position selected from said plurality of probe positions.

5. The system according to claim 2 wherein said position control unit is adapted to restrict the moving of said magnetic probe within an allowed spatial envelope.

6. The system according to claim 2 wherein said open magnetic probe is selected from a U-shaped open magnetic probe, a C-shaped open magnetic probe and a Y-shaped open magnetic probe.

7. The system according to claim 2 wherein said open magnetic probe is selected from an open magnetic probe comprising permanent magnets, an open yoked magnetic probe comprising permanent magnets, an open yoked magnetic probe comprising permanent magnets a ferromagnetic yoke and ferromagnetic collimators, an open magnetic probe comprising electromagnets, and a hybrid open magnetic probe including permanent magnets and electromagnets.

8. The system according to claim 7 wherein said electromagnets are selected from resistive electromagnets and superconducting electromagnets.

9. The system according to claim 1 wherein said positioning device is detachably attached to said operating table.

10. The system according to claim 1 wherein said operating table is positioned in an operating room, and said positioning device is attached to a part of said operating room, said part is selected from the ceiling of said operating room, at least one wall of said operating room, the floor of said operating room and any combination thereof.

11. The system according to claim 1 wherein each position of said plurality of probe positions is defined relative to a position selected from the position of said operating table and the position of said body part.

12. The system according to claim 1 wherein said positioning device is a transportable positioning device.

13. The system according to claim 1 wherein said plurality of probe positions comprises at least one probe position in which at least part of said magnetic probe is disposed under said operating table.

14. The system according to claim 1 wherein said plurality of probe positions comprises at least two probe positions suitable for performing magnetic resonance imaging of said body part to obtain at least two different partially overlapping images of said body part, said at least two partially overlapping images are suitable for being processed to obtain therefrom a composite image having an image volume larger than the image volume of each of said at least two partially overlapping images.

15. A position controlling device for use in a magnetic resonance imaging system for obtaining magnetic resonance images of a body part of a patient using an open magnetic probe, the device comprising:
a controllably movable framework attachable to said open magnetic probe for controllably positioning said open magnetic probe in a position selected from a plurality of probe positions, said plurality of positions includes at least one imaging position in which said open magnetic probe is positioned relative to said body part of said patient such that imaging procedures and surgical procedures can be performed on said body part of said patient while said open magnetic probe is positioned in said at least one imaging position; and
a position control unit operatively connected to said movable framework for controlling the moving of said open magnetic probe by said movable framework from one position of said plurality of probe positions to another position of said plurality of probe positions.

16. The device according to claim 15 wherein said movable framework is detachably attachable to an operating table supporting said patient.

17. The device according to claim 16 wherein said plurality of probe positions comprises at least one probe position in which said magnetic probe is at least partially disposed under said operating table.

18. The device according to claim 16 wherein said operating table is positioned in an operating room, and said positioning device is attached to a part of said operating rooms said part is selected from the ceiling of said operating room, at least one wall of said operating room, the floor of said operating room and any combination thereof.

19. The device according to claim 16 wherein each position of said plurality of probe positions is defined relative to a point positioned on said operating table and a point on or within said body part, said body part being attached to or disposed upon said operating table.

20. The device according to claim 15 wherein each position of said plurality of probe positions is defined relative to a point on or within said body part.

21. The device according to claim 15 wherein said plurality of probe positions comprises at least two probe positions suitable for performing magnetic resonance imaging of said body part to obtain at least two different partially overlapping images of said body part, said at least two partially overlapping images are suitable for being processed to obtain therefrom a composite image having an image volume larger than the image volume of each of said at least two partially overlapping images.

22. The device according to claim 15 wherein said movable framework is a motorized framework comprising at least one motor operatively coupled to said framework for moving said magnetic probe with at least 1 degree of freedom.

23. The device according to claim 15 wherein said position control unit comprises a microprocessor in communication with a data storage unit for retrievably storing data representative of said plurality of probe positions and for controlling the moving of said magnetic probe into a probe position selected from said plurality of probe positions.

24. The device according to claim 16 wherein each position of said plurality of probe positions is defined relative to the position of said body part.

25. The device according to claim 15 wherein said position control unit is adapted to restrict the moving of said magnetic probe within an allowed spatial envelope.

26. The device according to claim 15 wherein said positioning device is a transportable positioning device.

27. The device according to claim 15 wherein said movable framework is attached to a part of an operating room, said part is selected from the ceiling of said operating room, at least one wall of said operating room, the floor of said operating room and any combination thereof.

28. A controllably movable magnetic probe device for use in a magnetic resonance imaging system for obtaining magnetic resonance images of a body part, the device comprising:
an open magnet for producing a volume of substantially homogenous magnetic field for imaging a body part, said magnet is adapted for being disposed in at least one imaging position relative to said body part such that surgical procedures and imaging procedures can be sequentially or simultaneously performed on said body part while said open magnet is positioned in said at least one imaging position; and
a controllable positioning device attached to said open magnet for controllably IS positioning said open magnet in a position selected from a plurality of magnet positions, said plurality of positions includes said at least one imaging position.

29. The device according to claim 28 wherein each position of said plurality of magnet positions is defined relative to a point on or within said body part.

30. The device according to claim 28 wherein said plurality of magnet positions comprises at least two magnet positions suitable for performing magnetic resonance imaging of said body part to obtain at least two different partially overlapping images of said body part, said at least two partially overlapping images are suitable for being processed to obtain therefrom a composite image having an image volume larger than the image volume of each of said at least two partially overlapping images.

31. The device according to claim 28 wherein said movable framework is a motorized framework comprising at least one motor operatively coupled to said framework, for moving said magnet with at least 1 degree of freedom.

32. The device according to claim 28 wherein each position of said plurality of magnet positions is defined relative to the position of said body part.

33. The device according to claim 28 wherein said position control unit is adapted to restrict the moving of said magnetic probe within an allowed spatial envelope.

34. The device according to claim 28 wherein said allowed spatial envelope is defined with respect to an operating table attached to said controllable positioning device.

35. The device according to claim 28 wherein said positioning device is a transportable positioning device.

36. The device according to claim 28 wherein said positioning device comprises a controllably movable framework attached to said open magnet and a position control unit operatively connected to said movable framework for controlling the moving of said open magnet by said movable framework from one position of said plurality of magnet positions to another position of said plurality of magnet positions.

37. The device according to claim 36 wherein said movable framework is attached to a part of an operating room, said part is selected from the ceiling of said operating room, at least one wall of said operating room, the floor of said operating room and any combination thereof.

38. The device according to claim 36 wherein said position control unit comprises a microprocessor in communication with a data storage unit for retrievably storing data representative of said plurality of magnet positions and for controlling the moving of said magnet into a magnet position selected from said plurality of magnet positions.

39. The device according to claim 35 wherein said movable framework is detachably attachable to an operating table.

40. The device according to claim 39 wherein each position of said plurality of magnet positions is defined relative to a point positioned on said operating table and a point on or within said body part, said body part being attached to or disposed upon said operating table.

41. The system according to claim 39 wherein said plurality of magnet positions comprises at least one magnet position in which said magnet is at least partially disposed under said operating table.

42. The device according to claim 39 wherein said operating table is positioned in an operating room, and said positioning device is attached to a part of said operating room, said part is selected from the ceiling of said operating room, at least one wall of said operating room, the floor of said operating room and any combination thereof.
